(19) United States
(12) Patent Application Publication
(10) Pub. No.: US 2010/0120653 A1
(43) Pub. Date: May 13, 2010

(54) DETERGENT COMPOSITION FOR CLOTHING

(75) Inventors: Takanori Kotera, Wakayama-shi (JP); Daiya Murata, Wakayama-shi (JP); Akira Saito, Wakayama-shi (JP); Atsushi Nagasawa, Wakayama-shi (JP)

Correspondence Address: BIRCH STEWART KOLASCH & BIRCH, PO BOX 747, FALLS CHURCH, VA, 22040-0747, US

(73) Assignee: Kao Corporation, Tokyo (JP)

(21) Appl. No.: 12/594,677

(22) PCT Filed: Apr. 4, 2008
(86) PCT No.: PCT/JP2008/057117

§ 371 Date: Oct. 29, 2009

(30) Foreign Application Priority Data
Apr. 6, 2007 (JP) 2007-100252

(51) Int. Cl. C11D 1/68; C07C 43/10

(52) U.S. Cl. 510/356; 568/678

(57) ABSTRACT

The present invention is a detergent composition for clothing, containing (a) monoethers of glycerol and/or polyglycerols each represented by the formula (I) [hereinafter, referred to as component (a)]:

R—O—(C₃H₆O₂)ₙ—H (I)

(wherein, R represents a hydrocarbon group having 6 to 22 carbon atoms; and n represents a degree of glycerol condensation ranging from 1 to 7),
wherein the component (a) contains compounds of the formula (I) having different degrees of glycerol condensation n, and not less than 40% by mass of the component (a) is compounds in which R's are alkyl groups having 12 and/or 14 carbon atoms
and degrees of glycerol condensation n's are 3 to 5.

### FIELD OF THE INVENTION

The present invention relates to a detergent composition for clothing.

### BACKGROUND OF THE INVENTION

Use of nonionic surfactants such as monoalkylethers of glycerol and polyglycerol using glycerol produced mainly from plant-derived natural oil-and-fat as a raw material for increasing detergency have recently been disclosed in JP-A2001-49290, JP-A2001-49291, JP-A11-310792, JP-A4-506367, JP-A7-500861, JP-A3-174496, and JP-A2006-348084.

### SUMMARY OF THE INVENTION

The present invention relates to a detergent composition for clothing, containing (a) monoethers of glycerol or polyglycerols each represented by the formula (I) [hereinafter, referred to as component (a)]:

R—O—(C₃H₆O₂)ₙ—H (I)

(wherein, R represents a hydrocarbon group having 6 to 22 carbon atoms; and n represents a degree of glycerol condensation of 1 to 7),
wherein the component (a) contains compounds of the formula (I) having different n's, and not less than 40% by mass of the component (a) is compounds in which R's are alkyl groups having 12 and/or 14 carbon atoms and n's are 3 to 5.

### DETAILED DESCRIPTION OF THE INVENTION

Monoethers of glycerol and polyglycerol disclosed in above-described patent documents are not wholly-satisfactory in detergency, when used in detergent compositions for clothing. Particularly at low temperature, these exhibit high crystallinity and has low solubility in water, resulting in tendency of decreased detergency. The present inventors have investigated and found that a degree of glycerol condensation and a distribution thereof have large effects on detergency.

The present invention provides a detergent composition for clothing having increased detergency, containing monoethers of glycerol and/or polyglycerols (hereinafter, also referred to as (poly)glycerol monoethers) having a specific degree of glycerol condensation.

There is also a demand for a component that does not increase carbon dioxide in the air, or a carbon-neutral component, from the viewpoint of carbon cycle including increase of carbon dioxide. In such circumstance, (poly)glycerol monoethers such as the component (a) offer promising prospects, as these are produced by a process that will not increase carbon dioxide.

According to the present invention, a detergent composition for clothing having good detergency is provided.

<Component (a)>

The component (a) of the present invention contains the (poly) glycerol monoethers each produced by replacing a hydrogen atom of a hydroxy group in glycerol or a polyglycerol, a condensate of glycerol, with a hydrocarbon group having 6 to 22 carbon atoms to form an ether bond.

In the component (a), a total amount of polyglycerol monoethers in which R's represent alkyl groups having 12 and/or 14 carbon atoms and degrees of glycerol condensation n's are 3 to 5 is not less than 40% by mass, preferably not less than 50% by mass, more preferably not less than 60% by mass, even more preferably not less than 70% by mass, and still even more preferably not less than 80% by mass. From the viewpoint of detergent performance at low temperature, the upper limit of the amount is preferably 99% by mass, more preferably 95% by mass, even more preferably 90% by mass, and still even more preferably 85% by mass. From the viewpoint of detergent performance at low temperature, the component (a) preferably contains compounds represented by the formula (I) having different degrees of glycerol condensation n's, preferably two or more n's, particularly preferably three or more n's. In the component (a), compounds in which R's represent alkyl groups having 12 and/or 14 carbon atoms and degrees of glycerol condensation n's are 3 to 5 exhibit a highest detergent performance. However, when the component (a) contains compounds having the same degree of glycerol condensation n, though satisfying the conditions above, it is easy to crystallize and has decreased solubility in water particularly at low temperature, resulting in a tendency of decreased detergency. In contrast, when the component (a) contains compounds having different degrees of glycerol condensation n's, it is not easy to crystallize and has high solubility in water at low temperature, resulting in good detergent performance. The component (a) thus preferably contains at least two, and more preferably whole three compounds having different degrees of glycerol condensation n's of 3 to 5 (n=3, 4, 5). Further, the component (a) containing polyglycerol monoethers in which R's represent alkyl groups having 12 and/or 14 carbon atoms and degrees of glycerol condensation n's are 3 to 5 in an amount of not more than 99% by mass in total has significantly increased solubility at low temperature, resulting in largely increased detergent performance. In general, a smaller content of the polyglycerol monoether results in a higher solubility at low temperature but also results in a lower detergent performance at ambient temperature. Therefore, the content is required to be at moderate balance. When the detergent composition is in the liquid form, it can prevent separation during storage and hold its commercial value in storage for long periods.

The component (a) of the present invention preferably contains compounds (a-1) in which each R of the formula (I) in the component (a) represents an alkyl group having 12 carbon atoms and each degree of glycerol condensation n is 3 to 5 and compounds (a-2) in which each R of the formula (I) represents an alkyl group having 14 carbon atoms and each degree of glycerol condensation n is 3 to 5 in the total amount of not less than 40% by mass, more preferably contains compounds having different n's, and particularly preferably contains three compounds having n=3, 4, and 5 selected from compounds (a-1) and (a-2).

From the viewpoint of detergency, a degree of glycerol condensation n of a raw material for the component (a) is preferably 4. Among (poly)glycerol ethers having degrees of glycerol condensation of 1 to 7, a total amount of polyglycerol monoethers having a degree of glycerol condensation n of 4 is preferably not less than 10% by mass, more preferably not less than 15% by mass, even more preferably not less than 20% by mass, and still even more preferably not less than 30% by mass.

In the component (a), a total amount of (poly)glycerol monoethers having degrees of glycerol condensation n's of 1 and 2 is preferably less than 50% by mass, and more preferably not more than 35% by mass. Further, in the component (a), a content of glycerol monoethers having a degree of glycerol condensation n of 1 is less than 30% by mass, and more preferably not more than 20% by mass.

In the formula (I), R is preferably an alkyl group having 6 to 22, more preferably 12 to 14, and particularly preferably 12 carbon atoms, which may be linear, branched, saturated, or unsaturated. In the component (a), or in the total amount of compounds of the formula (I) each having a degree of glycerol condensation n of 1 to 7, a total amount of compounds of the formula (I) in which R's represent alkyl groups having 12 to 14 carbon atoms, particularly 12 and 14 carbon atoms is preferably not less than 40% by mass, more preferably not less than 70% by mass, even more preferably not less than 90% by mass, and still even more preferably not less than 95%.

In the formula (I), a condensed glycerol moiety is represented as (C₃H₆O₂)ₙ. The representation not only shows a linear form, but also includes a branched form and a randomly mixed form of a linear form and a branched form. It should be noted that the representation is for convenience.

A constitution of the component (a) by degree of glycerol condensation on bases of mass [mass percent in the component (a)] can be determined from area percentages according to Gas Chromatography (GC).

The component (a) of the present invention can also be prepared by, for example, reacting an alcohol having 6 to 22 carbon atoms with 2,3-epoxy-1-propanol (glycidol) in a predetermined amount in the presence of an alkali catalyst, or by a method described in paragraphs 0007 to 0011 of JP-A2000-160190.

A bonding mode of glycerol in the component (a) may be any form, including a linear form (bonding of glycerol at 1- and 3-positions) and a branched form (bonding of glycerol at 1- and 2-positions, and further bonding at 1- and/or 3-position of glycerol bonded at 2-position).

In general, (poly)glycerol monoethers such as the component (a) are prepared as a mixture of compounds having different condensation degrees. From the viewpoint of detergency, the present invention uses a mixture containing compounds having degrees of glycerol condensation ranging from 3 to 5 in a specific ratio. Such a compound having a degree of condensation within the range can be obtained by purifying a reactant, for example, by distillation, according to need.

<Component (b)>

The detergent composition for clothing of the present invention can further contain an alkali agent [hereinafter, also referred to as component (b)]. Examples of the component (b) include, when the detergent composition is in a powder form, carbonates, bicarbonates, silicates, orthosilicates, metasilicates, crystalline silicates, and phosphates. Salts are preferably alkaline metal salts such as sodium salts and potassium salts. These alkali agents may be used alone or as a mixture of two or more agents. Specific examples of the alkali agent include sodium carbonate, potassium carbonate, sodium hydrogen carbonate, sodium silicate No. 1, sodium silicate No. 2, sodium silicate No. 3, sodium tetraborate, sodium pyrophosphate, and sodium tripolyphosphate. As used herein, the crystalline silicate refers an alkali substance that produces 0.1% by mass dispersant having the maximum pH of not less than 11 in ion-exchanged water at 20° C. and requires not less than 5 ml of 0.1N—HCl aqueous solution to reduce the pH of 1 L of the dispersant to 10. The crystalline silicate is distinguished from a zeolite (crystalline aluminosilicate) as a component (c) described below. The crystalline silicate is preferably in a lamellar form. Those can be used, described in JP-A7-89712, JP-A60-227895, Phys. Chem. Glasses. 7, p 127-p 138 (1966), and Z. Kristallogr., 129, p 396-p 404 (1969), for example. A crystalline silicate represented by the formula 0.42Na₂O.0.14K₂O.SiO₂.0.03CaO.0.0005MgO is preferably used. Powder and granules of crystalline silicate are also commercially available from Hoechst, which are called trade name “Na-SKS-6” (δ-Na₂Si₂O₅). When the detergent composition is in the form of liquid, examples of the component (b) used include alkanolamines such as monoethanolamine, diethanolamine, triethanolamine, methylmonoethanolamine, dimethylethanolamine, and 3-aminopropanol; and inorganic salts such as sodium hydroxide, potassium hydroxide, sodium silicate, and sodium carbonate. The component (b) is particularly preferably at least one selected from monoethanolamine, sodium hydroxide, and potassium hydroxide.

A pH of the detergent composition for clothing of the present invention is preferably 7 to 14, more preferably 8 to 12, and even more preferably 9 to 11, when diluted in ion-exchanged water to a concentration of 0.1% by mass.

<Component (c)>

The detergent composition for clothing of the present invention can further contain (c) a zeolite [hereinafter, also referred to as component (c)]. In the present invention, the zeolite as the component (c) is a crystalline aluminosilicate preferably represented by the formula (cl) and more preferably the formula (c2).

a(M₂O).Al₂O₃.b(SiO₂).w(H₂O) (c1)

(wherein, M represents an alkaline metal atom; a, b, and w represent molar ratios of components, respectively, generally satisfying 0.7≦a≦1.5, 0.8≦b≦6, and w being an arbitrary positive number).

Na₂O.Al₂O₃.n(SiO₂).m(H₂O) (c2)

(wherein, n represents the number of 1.8 to 3; and m represents the number of 1 to 6).

Examples of the component (c) include synthetic zeolites such as A, X, and P zeolites. A preferred average particle diameter of the component (c) is 0.1 to 10 μm.

<Component (d)>

The detergent composition of the present invention preferably contains an alcohol having 6 to 22 carbon atoms as a component (d). Combination use of the component (d) with the component (a) enhances anti-crystallization of the component (a) and can further increase effects of increasing detergent performance at low temperature. An amount of the component (d) used is preferably 0.001 to 20% by mass, more preferably 0.001 to 10% by mass, and even more preferably 0.1 to 10% by mass to the component (a). The composition containing the component (d) in an amount of more than 20% by mass tends to have decreased detergent performance due to the component (d) itself acting as pollution.

The component (d) is preferably an alcohol having an alkyl group of 6 to 22 carbon atoms. The alkyl group may be linear or branched. The alcohol is particularly preferably 1-decanol, 1-dodecanol, or 1-tetradecanol.

<Component (e)>

The detergent composition of the present invention can further contain at least one compound selected from glycerol and polyglycerols as a component (e). Combination of the component (e) with the component (a) also enhances anti-crystallization of the component (a), which is preferred from the viewpoint of increasing detergent performance at low temperature. When the detergent composition of the present invention is in the liquid form, the combination use also decreases viscosity of the detergent composition, resulting in good metric properties. An amount of the component (e) used is preferably 0.001 to 50% by mass, more preferably 0.001 to 20% by mass, even more preferably 0.1 to 10% by mass, and still even more preferably 1 to 5% by mass to the component (a).

The component (e) is preferably glycerol and/or a polyglycerol. Any polyglycerol can be used as the component (e) without specific limitation in condensation degree and binding mode. A condensation degree of the polyglycerol ranges from 2 to 8. The polyglycerol may be of a linear or cyclic form.

<Component (f)>

The detergent composition of the present invention preferably further contains at least one surfactant as a component (f) selected from (f-1) alkyl sulfates and/or polyoxyethylene alkyl ether sulfates (10 to 18, preferably 12 to 14 carbon atoms), in which each alkyl group has 10 to 18 carbon atoms, and preferably 12 to 14 carbon atoms [hereinafter, also referred to as component (f-1)]; (f-2) α-sulfofatty acid ester salts, in which an alkyl group has 14 to 18 carbon atoms, and preferably 16 to 18 carbon atoms [hereinafter, also referred to as component (f-2)]; and (f-3) fatty acid salts [hereinafter, also referred to as component (f-3)].

<Component (f-1)>

In the detergent composition of the present invention, combination use of the component (a) with the component (f-1) generates a synergistic effect on detergency to provide detergent performance that cannot achieve with respective components alone. A ratio of the component (a) to the component (f-1) is, represented by (a)/(f-1) of mass ratio, preferably 1/9 to 9/1, more preferably 2/8 to 8/2, even more preferably 3/7 to 7/3, and still even more preferably 4/6 to 6/4. In the polyoxyethylene alkyl ether sulfates, an average addition mole number of ethylene oxide is preferably 0.5 to 5.0. For the component (f-1), preferred are decyl sulfates, dodecyl sulfates, tetradecyl sulfates, and polyoxyethylene decyl ether sulfates, polyoxyethylene dodecyl ether sulfates, and polyoxyethylene tetradecyl ether sulfates, each having an average addition mole number of ethylene oxide of 1 to 3. Preferred examples of a counter ion of these salts include sodium, potassium, and ammonium.

<Component (f-2)>

In the detergent composition of the present invention, combination use of the component (a) with the component (f-2) improves solubility particularly at low temperature, so that a synergistic effect on detergency at low temperature is generated to provide detergent performance that cannot achieve with respective components alone. A ratio of the component (a) to the component (f-2) is, represented by (a)/(f-2) on bases of mass, preferably 1/9 to 9/1, more preferably 2/8 to 8/2, even more preferably 3/7 to 7/3, and still even more preferably 4/6 to 6/4.

An ester group of the component (f-2) is preferably a methyl, ethyl, or propyl group, and particularly preferably a methyl group. Preferred counter ions of these salts are sodium and potassium. Particularly preferred is sodium.

<Component (f-3)>

In the detergent composition of the present invention, combination use of the component (a) with a fatty acid salt as the component (f-3) increases effects of defoaming, because metal soap generated by reaction of the component (f-3) with hard components in washing water is more finely dispersed due to the component (a) than a general surfactant. An amount of the fatty acid salt thus can be decreased. A ratio of the component (a) to the component (f-3) is, represented by (a)/(f-3) of mass ratio, preferably 1000/1 to 1/10, more preferably 100/1 to 1/1, even more preferably 50/1 to 2/1, and still even more preferably 10/1 to 3/1. The component (f) is preferably a fatty acid salt having 12 to 22 carbon atoms. Specific examples of a fatty acid include lauric acid, myristic acid, palmitic acid, stearic acid, and oleic acid. Preferred counter ions of these salts are sodium and potassium. Particularly preferred is sodium.

### <Other Components>

The detergent composition for clothing of the present invention can further contain a surfactant other than the components (a), (f-1), (f-2), and (f-3). Examples of the other surfactant include anionic, nonionic, amphoteric and cationic surfactants and mixtures thereof. Preferably used are anionic and nonionic surfactants.

Examples of the anionic surfactant include sulfates of alcohol alkoxylates having 8 to 20 carbon atoms, alkylbenzenesulfonates, alkylsulfates, paraffin sulfonates, α-olefin sulfonates, α-sulfofatty acid salts, and α-sulfofatty acid alkyl esters. In the present invention, in order to increase detergent performance at low temperature, preferred are alkylbenzenesulfonates having a linear alkyl chain of 10 to 14 carbon atoms, and more preferably 12 to 14 carbon atoms. Preferred counter ions thereof are alkaline metals and amines. Particularly preferred are sodium and/or potassium, monoethanolamine, and diethanolamine.

Preferred examples of the nonionic surfactant include polyoxyalkylene alkyl (8 to 20 carbon atoms) ethers, alkyl polyglycosides, polyoxyalkylene alkyl (8 to 20 carbon atoms) phenyl ethers, polyoxyalkylene sorbitan fatty acid (8 to 22 carbon atoms) esters, polyoxyalkylene glycol fatty acid (8 to 22 carbon atoms) esters, and polyoxyethylene/polyoxypropylene block polymers. In order to increase detergent performance, particularly preferred for the nonionic surfactant are polyoxyalkylene alkyl ethers produced by adding 4 to 20 mol of alkylene oxide such as ethylene oxide and propylene oxide to an alcohol having 10 to 18 carbon atoms [e.g., those having an HLB value of 10.5 to 15.0, and preferably 11.0 to 14.5 (calculated by the Griffin's method)].

The detergent composition for clothing of the present invention can further contain an organic builder and/or an inorganic builder other than the components (b) and (c). Examples of the organic builder include carboxylates such as aminocarboxylates, hydroxyaminocarboxylates, hydroxycarboxylates, cyclocarboxylates, maleic acid derivatives and oxalates, and organocarboxylic acid (salt) polymers such as acrylic acid polymers and copolymers, polycarboxylic acid polymers and copolymers, glyoxylic acid polymers, polysaccharides and salts thereof. Organocarboxylic acid (salt) polymers are particularly preferred. For salts of these builders, a counter ion is preferably an alkaline metal or an amine, and particularly preferably a sodium or potassium, monoethanolamine, or diethanolamine. These builders may be used alone or in combination.

Particularly when a carboxylic acid (salt) polymer is contained in the detergent composition of the present invention, high affinity of the polymer and the component (a) provides following effects. In the detergent composition in a powder form, water absorption of the polymer can be suppressed. The detergent composition thus can contain the polymer at high ratio with keeping anti-caking properties of detergent particles, resulting in significantly increased detergent performance. In the detergent composition in a liquid form, the component (a) exhibits a suppressing effect of precipitation of the carboxylic acid (salt) polymer,resulting in increased storage stability.

The detergent composition for clothing of the present invention can further contain other additives such as a bleach (e.g., a percarbonate, a perborate, a bleaching activator), an anti-depositing agent (e.g., carboxymethylcellulose), a softener (e.g., a dialkyl type quaternary ammonium salt, clay mineral), a reducing agent (e.g., a sulfite), a fluorescent bleaching agent (e.g., a biphenyl type, an aminostilbene type), a foam-controlling agent (e.g., silicone), a flagrance, and an enzyme (e.g., protease, cellulase, pectinase, amylase, lipase).

When a biphenyl or aminostilbene fluorescent bleaching agent is contained in detergent composition of the present invention, since these fluorescent bleaching agents have low solubility to the component (a), the amount of the fluorescent bleaching agent taken into micelle of the surfactant is reduced, resulting in an increased adherence of the fluorescent bleaching agent to laundry. An amount of the fluorescent bleaching agent formulated in detergent composition thus can be decreased. According to the same mechanism, a flagrance, particularly a flagrance having a cLogP of not less than 3 dissolves in micelle of the surfactant in a decreased amount, resulting in increased scent of the flagrance adhering to and remaining on laundry and decreased change of a scent tone during and after washing. According to the same mechanism, a silicone can be adsorbed on laundry in an increased amount.

When an enzyme is contained in detergent composition of the present invention, since the component (a) has low enzyme activity-inhibitory rate, reduction of enzyme activity during storage can be prevented.

When the composition is in the form of granule, from the viewpoints of fluidity and anti-caking properties of the composition, it may be subjected to surface modification. For a surface modifier, the component (c) can be used. Examples of other surface modifier include silicate compounds such as calcium silicate, silicon dioxide, bentonite, talc, clay, amorphous silica derivatives, and crystalline silicates, metal soap, fine powders such as powdery surfactant, water-soluble polymers such as carboxymethylcellulose, polyethylene glycol, sodium polyacrylate, copolymers of acrylic acid and maleic acid and salts thereof, and other polycarboxylates, and fatty acids. Preferably used are the component (c) and crystalline silicates, and more preferably the component (c).

When the composition is in the form of granule, combination use of the component (a) and polyethylene glycol can increase fluidity in granulation to decrease generation of fine powder. This enables to reduce scattering of powder and increase anti-caking properties.

### <Detergent Composition for Clothing>

The detergent composition for clothing of the present invention preferably contains the component (a) in an amount of 1 to 80% by mass, more preferably 3 to 40% by mass, and even more preferably 5 to 20% by mass. The detergent composition preferably contains the component (b) in an amount of 1 to 90% by mass, more preferably 5 to 50% by mass, and even more preferably 10 to 40% by mass. The detergent composition preferably contains the component (c) in an amount of 1 to 90% by mass, more preferably 5 to 50% by mass, and even more preferably 10 to 40% by mass.

An amount of the component (d) to the component (a) in the composition is preferably 0.001 to 20% by mass, more preferably 0.01% to 10% by mass, and even more preferably 0.1 to 5% by mass. An amount of the component (e) to the component (a) in the composition is preferably 0.001 to 50% by mass, more preferably 0.01 to 20% by mass, even more preferably 0.01% to 10% by mass, and still even more preferably 0.05 to 5% by mass.

A content of other surfactants than the component (a) in the composition is preferably 0.1 to 50% by mass, more preferably 3 to 30% by mass, and even more preferably 5 to 15% by mass. A content of particularly the components (f-1) and (f-2) in the composition is preferably 3 to 30% by mass, more preferably 5 to 20% by mass, and even more preferably 5 to 15% by mass. A content of particularly the component (f-3) in the composition is preferably 0.1 to 15% by mass, more preferably 1 to 10% by mass, and even more preferably 1 to 5% by mass.

The detergent composition for clothing of the present invention is preferably in the form of powder having a bulk density of 300 to 1000 g/L, more preferably 500 to 900 g/L, and even more preferably 600 to 800 g/L, and having an average particle diameter of 150 to 3000 μm, more preferably 500 to 1500 μm, and even more preferably 600 to 1200 μm.

### Examples

The following Examples demonstrate the present invention. Examples are intended to illustrate the present invention and not to limit the present invention.

The following components and components shown in Table 1 were used to prepare powder detergent compositions for clothing shown in Tables 1 and 2. These compositions were evaluated for detergency according to the following method. Results are shown in Tables 1 and 2.

### [1] Components

<(poly)glycerol monoethers (1)>

In a 300 mL four-neck flask, under a reduced pressure of 25 kPa, 93.2 g (0.50 mol) of lauryl alcohol and 7.01 g (0.10 mol) of potassium methoxide were stirred and heated to 95° C. to distill methanol off. Under nitrogen flow, to this was added 148.16 (2.0 mol) of glycidol dropwise for 24 hours at 95° C., and stirred for additional 2 hours under the same conditions. After the end of the reaction, to the mixture was added 4.90 g (0.05 mol) of sulfuric acid and 10 g of water to neutralize a catalyst to give 248.1 g of reaction intermediate (conversion of glycidol: not less than 99.9%). Gas chromatography analysis of the reaction intermediate showed the presence of polyglycerol lauryl ethers where a percentage of ethers having degrees of glycerol condensation n's of 3 to 5 in the total of ethers having n's of 1 to 7 was 28.6% by mass. The reaction intermediate was subjected to distillation under a reduced pressure of 40 Pa and 210° C. to give a distilled product. Gas chromatography analysis of the resultant reaction product did not show the presence of lauryl alcohol, lauryl monoglycerol ether, lauryl diglycerol ether, glycerol or polyglycerol. In the resultant lauryl polyglycerol ethers, a percentage of ethers having degrees of glycerol condensation n's of 3 to 5 in the total of ethers having n's of 1 to 7 was 55.4% by mass. The presence of compounds having different n's in the product [(poly)glycerol monoethers (1)] was thus confirmed.

<(poly)glycerol monoethers (2)>

In a 300 mL four-neck flask, under nitrogen flow, 93.2 g (0.50 mol) of lauryl alcohol and 2.94 g (0.0050 mol) of lanthanum triflate were stirred and heated to 90° C. To this was added 148.16 g (2.0 mol) of glycidol dropwise for 24 hours at the same temperature, and stirred for additional 2 hours under the same conditions to give 251.5 g of reaction product. Gas chromatography analysis of the reaction product showed that a conversion of glycidol was not less than 99.9% and contents of lauryl alcohol and polyglycerol were 6.0% by mass and 2.2% by mass respectively. The analysis also showed that in the resultant lauryl (poly)glycerol ethers, a percentage of ethers having degrees of glycerol condensation n's of 3 to 5 in the total of ethers having n's of 1 to 7 was 43.3% by mass. The presence of compounds having different n's in the product [(poly)glycerol monoethers (2)] was thus confirmed.

<(poly)glycerol monoethers (a1) to (a13)>

The reaction intermediate produced during the production of the (poly)glycerol monoethers (1) was subjected to column separation to fractionate components (a1) to (a7). These components were measured for molecular weight by Mass spectrometry. These components were used alone or in combination as shown in Table 2.

(poly)glycerol monoether (a1): molecular weight of not less than 220 and less than 300 (corresponding to a degree of glycerol condensation n=1)

(poly)glycerol monoether (a2): molecular weight of not less than 300 and less than 360 (corresponding to a degree of glycerol condensation n=2)

(poly)glycerol monoether (a3): molecular weight of not less than 360 and less than 440 (corresponding to a degree of glycerol condensation n=3)

(poly)glycerol monoether (a4): molecular weight of not less than 440 and less than 520 (corresponding to a degree of glycerol condensation n=4)

(poly)glycerol monoether (a5): molecular weight of not less than 520 and less than 600 (corresponding to a degree of glycerol condensation n=5)

(poly)glycerol monoether (a6): molecular weight of not less than 600 and less than 680 (corresponding to a degree of glycerol condensation n=6)

(poly)glycerol monoether (a7): molecular weight of not less than 680 and not more than 760 (corresponding to a degree of glycerol condensation n=7)

Each of following higher alcohols was reacted with the four times molar amount of glycidol in the presence of an alkali catalyst to give polyglyceryl alkyl ethers. The product was subjected to column separation to collect only a component having a degree of glycerol condensation of 4. Components thus obtained were used alone or in combination as shown in Table 2.

(poly)glycerol monoether (a8): C₆H₁₃OH

(poly)glycerol monoether (a9): C₁₀H₂₁OH

(poly)glycerol monoether (a10): C₁₄H₂₉OH

(poly)glycerol monoether (a11): C₁₆H₃₃OH

(poly)glycerol monoether (a12): C₁₈H₃₇OH

(poly)glycerol monoether (a13): C₂₂H₄₅OH

### <AS>

For AS, sodium tetradecyl sulfate (Kao Corporation) was used.

### <LAS>

For LAS, NEOPELEXG-15(Kao Corporation) was used.

### <Zeolite>

For zeolite, a 4A zeolite having an average particle diameter of 3 μm (Tosoh Corporation) was used.

### <Lauryl Alcohol>

For lauryl alcohol, Kalcol 2098 (Kao Corporation) was used.

### <Diglycerol>

For diglycerol, a reagent (Wako Pure Chemical Industries, Ltd.) was used.

### [2] Method for Evaluating Detergency

To 1 L each of tap water were added 0.6667 g each of detergent compositions shown in Tables 1 and 2 and dissolved. To these each were added five pieces of cloth stained with spinach, which was prepared as described below, and washed for 10 minutes with a Terg-O-Tometer at 80 round/min and 20° C. (liquid temperature). Test pieces were sufficiently rinsed and dried. A washing rate was determined according to the following formula.

$$Washing rate ( \% ) = \frac{( \begin{matrix}reflectance after washing - \\ reflectance before washing\end{matrix} )}{( \begin{matrix}reflectance of clean cloth - \\ reflectance before washing\end{matrix} )} \times 100$$

Evaluation for detergency at low temperature was similarly conducted as above, except that a washing temperature was 5° C. (liquid temperature).

A reflectance was measured using NDR-10DP manufactured by Nippon Denshoku Industries Co., Ltd. with a 460 nm filter.

<Preparation of Cloth Stained with Spinach>

Commercially available spinach was pureed with a blender. A liquid part of the puree was filtered through cotton cloth. 0.5 g of the resultant liquid was uniformly applied on 6 cm by 6 cm of cotton test cloth #2023 and dried for 12 hours at 20° C. The dried cloth was used in the test.

| | | | | Example | | | | |
|---|---|---|---|---|---|---|---|---|
| | | | | 1-1 | 1-2 | 1-3 | 1-4 | 1-5 |
| Powdery | Compounding | (a) | (poly)glycerol monoethers(1) | 10 | 10 | | 10 | 10 |
| detergent | component | | (poly)glycerol monoethers(2) | | | 10 | | |
| composition | (mass %) | (b) | Sodium carbonate | 20 | 20 | 20 | 20 | 20 |
| | | (c) | Zeolite | 30 | 30 | 30 | 30 | 30 |
| | | (d) | Lauryl alcohol (separately added) | | 0.5 | | 0.5 | |
| | | (e) | Diglycerol (separately added) | | 0.2 | | | 0.2 |
| | | (f) | AS | 10 | 10 | 10 | 10 | 10 |

| | | | | | |
|---|---|---|---|---|---|
| Sodium sulfate | Balance | Balance | Balance | Balance | Balance |
| Total | 100 | 100 | 100 | 100 | 100 |

| | | | | | |
|---|---|---|---|---|---|
| Ratio of component(d) | 0 | 5 | 6.0 | 5 | 0 |
| [to the component(a), % by weight] *^{a} | | | | | |
| Ratio of component(e) | 0 | 2 | 2.2 | 0 | 2 |
| [to the component(a), % by weight] *^{b} | | | | | |

| | | | | | |
|---|---|---|---|---|---|
| Detergency(20° C.) (%) | 82 | 80 | 78 | 79 | 81 |
| Detergency(5° C.) (%) | 51 | 61 | 59 | 55 | 57 |

*^{a} the amount of the component (d) was calculated from the total amount of lauryl alcohol derived from (poly)glycerol monoethers (2) and lauryl alcohol separately added.
*^{b} an amount of the component (e) was calculated from the total amount of diglycerol derived from (poly)glycerol monoethers (2) and diglycerol separately added.

TABLE 2

| | | | | Example | | | | | | | | | | | Comparative example | | | |
|---|---|---|---|---|---|---|---|---|---|---|---|---|---|---|---|---|---|---|
| | | | | 2-1 | 2-2 | 2-3 | 2-4 | 2-5 | 2-6 | 2-7 | 2-8 | 2-9 | 2-10 | 2-11 | 1 | 2 | 3 | 4 |
| Powdery | Com- | (poly)glyc- | (a1) | | 1.5 | | 0.8 | | | | | | 3 | 1 | 5 | 4.5 | 4.2 | 20 |
| deter- | pounding | erol | (a2) | | 1.5 | | 1.4 | | | | | | 5.5 | 5.1 | | 3.5 | 3.5 | |
| gent com- | component | monoether | (a3) | | | 5.4 | 5.1 | 10 | 5 | 5 | 5 | 10 | 3.3 | 4.3 | | | 2.2 | |
| position | (mass %) | | (a4) | 15 | 17 | 5.8 | 6.2 | 5 | 10 | 5 | 5 | 10 | 3.5 | 4.5 | 7 | 6 | 2.1 | |
| | | | (a5) | | | 4.6 | 4.8 | 5 | 5 | 10 | | | 2.6 | 3 | | | 1.9 | |
| | | | (a6) | 3 | | 2.2 | 1 | | | | | | 1.4 | 1.6 | 8 | 6 | 2.6 | |
| | | | (a7) | 2 | | 2 | 0.7 | | | | | | 0.7 | 0.5 | | | 3.5 | |
| | | | (a8) | | | | | | | | | | | | | | | |
| | | | (a9) | | | | | | | | | | | | | | | |
| | | | (a10) | | | | | | | | 10 | | | | | | | |
| | | | (a11) | | | | | | | | | | | | | | | |
| | | | (a12) | | | | | | | | | | | | | | | |
| | | | (a13) | | | | | | | | | | | | | | | |

| | | | | | | | | | | | | | | | |
|---|---|---|---|---|---|---|---|---|---|---|---|---|---|---|---|
| LAS | 15 | 15 | 15 | 15 | 15 | 15 | 15 | 15 | 15 | 15 | 15 | 15 | 15 | 15 | 15 |
| Sodium carbonate | 20 | 20 | 20 | 20 | 20 | 20 | 20 | 20 | 20 | 20 | 20 | 20 | 20 | 20 | 20 |
| Sodium sulfate | 15 | 15 | 15 | 15 | 15 | 15 | 15 | 15 | 15 | 15 | 15 | 15 | 15 | 15 | 15 |
| Zeolite | 30 | 30 | 30 | 30 | 30 | 30 | 30 | 30 | 30 | 30 | 30 | 30 | 30 | 30 | 30 |

| | | | | | | | | | | | | | | | |
|---|---|---|---|---|---|---|---|---|---|---|---|---|---|---|---|
| Ratio of (a3) + (a4) + (a5) *¹ | 75 | 85 | 79 | 80.5 | 100 | 100 | 100 | 100 | 100 | 47 | 59 | 35 | 30 | 31 | 0 |
| Ratio of (a1) + (a2) *² | 0 | 15 | 0 | 11 | 0 | 0 | 0 | 0 | 0 | 42.5 | 30.5 | 25 | 40 | 38.5 | 100 |
| Ratio of (a1) *³ | 0 | 7.5 | 0 | 4 | 0 | 0 | 0 | 0 | 0 | 15 | 5 | 25 | 22.5 | 21 | 100 |

| | | | | | | | | | | | | | | | |
|---|---|---|---|---|---|---|---|---|---|---|---|---|---|---|---|
| Detergency(20° C.) (%) | 74 | 72 | 69 | 70 | 74 | 76 | 73 | 72 | 71 | 62 | 65 | 40 | 36 | 38 | 5 |
| Detergency(5° C.) (%) | 56 | 59 | 55 | 57 | 61 | 62 | 61 | 60 | 58 | 54 | 55 | 32 | 28 | 29 | 4 |

| | | | | Comparative example | | | | | | | | | | |
|---|---|---|---|---|---|---|---|---|---|---|---|---|---|---|
| | | | | 5 | 6 | 7 | 8 | 9 | 10 | 11 | 12 | 13 | 14 | 15 |
| Powdery | Com- | (poly)glyc- | (a1) | | | | | | | | | | | |
| deter- | pounding | erol | (a2) | 20 | | | | | | | | | | |
| gent com- | component | monoether | (a3) | | 20 | | | | | | | | | |
| position | (mass %) | | (a4) | | | 20 | | | | | | | | 10 |
| | | | (a5) | | | | 20 | | | | | | | |
| | | | (a6) | | | | | | | | | | | |
| | | | (a7) | | | | | | | | | | | |
| | | | (a8) | | | | | 20 | | | | | | |
| | | | (a9) | | | | | | 20 | | | | | |
| | | | (a10) | | | | | | | 20 | | | | 10 |
| | | | (a11) | | | | | | | | 20 | | | |
| | | | (a12) | | | | | | | | | 20 | | |
| | | | (a13) | | | | | | | | | | 20 | |

| | | | | | | | | | | | |
|---|---|---|---|---|---|---|---|---|---|---|---|
| LAS | 15 | 15 | 15 | 15 | 15 | 15 | 15 | 15 | 15 | 15 | 15 |
| Sodium carbonate | 20 | 20 | 20 | 20 | 20 | 20 | 20 | 20 | 20 | 20 | 20 |
| Sodium sulfate | 15 | 15 | 15 | 15 | 15 | 15 | 15 | 15 | 15 | 15 | 15 |
| Zeolite | 30 | 30 | 30 | 30 | 30 | 30 | 30 | 30 | 30 | 30 | 30 |

| | | | | | | | | | | | |
|---|---|---|---|---|---|---|---|---|---|---|---|
| Ratio of (a3) + (a4) + (a5) *¹ | 0 | 100 | 100 | 100 | 100 | 100 | 100 | 100 | 100 | 100 | 100 |
| Ratio of (a1) + (a2) *² | 100 | 0 | 0 | 0 | 0 | 0 | 0 | 0 | 0 | 0 | 0 |
| Ratio of (a1) *³ | 0 | 0 | 0 | 0 | 0 | 0 | 0 | 0 | 0 | 0 | 0 |

| | | | | | | | | | | | |
|---|---|---|---|---|---|---|---|---|---|---|---|
| Detergency(20° C.) (%) | 12 | 74 | 77 | 72 | 58 | 67 | 72 | 69 | 68 | 63 | 75 |
| Detergency(5° C.) (%) | 9 | 46 | 48 | 44 | 29 | 33 | 42 | 33 | 32 | 31 | 48 |

cf.
*¹ a percentage by mass of the total amount of compounds having degrees of glycerol condensation n's of 3 to 5 and alkyl groups having 12 and/or 14 carbon atoms in the total amount of (a1) to (a13)
*² a percentage by mass of the total amount of compounds each having degrees of glycerol condensation n's of 1 to 2 in the total amount of (a1) to (a13)
*³ a percentage by mass of a compound having a degree of glycerol condensation n of 1 in the total amount of (a1) to (a13)

1. A detergent composition for clothing, comprising (a) monoethers of glycerol or polyglycerols each represented by the formula (I) [hereinafter, referred to as component (a)]:
R—O—(C₃H₆O₂)ₙ—H (I)
(wherein, R represents a hydrocarbon group having 6 to 22 carbon atoms; and n represents a degree of glycerol condensation ranging from 1 to 7),
wherein the component (a) comprises compounds of the formula (I) having different degrees of glycerol condensation n's, and not less than 40% by mass of the component (a) is compounds in which R's are alkyl groups having 12 and/or 14 carbon atoms and degrees of glycerol condensation n's are 3 to 5.

2. The detergent composition for clothing according to claim 1, wherein the component (a) comprises compounds having degrees of glycerol condensation n's of 1 or 2 in an amount of less than 50% by mass.

3. The detergent composition for clothing according to claim 1 or 2, wherein the component (a) comprises compounds having a degree of glycerol condensation n of 1 in an amount of less than 30% by mass.

4. The detergent composition for clothing according to claim 1 or 2, further comprising an alcohol having 6 to 22 carbon atoms in an amount of 0.001 to 20% by mass to the component (a).

5. The detergent composition for clothing according to claim 1 or 2, further comprising at least one compound selected from the group consisting of glycerol and polyglycerols in an amount of 0.001 to 50% by mass to the component (a).
